(19) United States
(12) Patent Application Publication
(10) Pub. No.: US 2025/0153205 A1
(43) Pub. Date: May 15, 2025

(54) CONCURRENT DISPERSION OF LIQUID COMPOSITIONS

(71) Applicant: MARKESBERY BLUE PEARL LLC, Hebron, KY (US)

(72) Inventors: W. Russell Markesbery, Hebron, KY (US); John E. Busse, Cincinnati, OH (US); James K. Busse, Cincinnati, OH (US); Daric Foster, Cincinnati, OH (US); Eugene J. Pancheri, Hebron, KY (US)

(21) Appl. No.: 18/836,568

(22) PCT Filed: Feb. 8, 2023
(86) PCT No.: PCT/US2023/012558

§ 371 Date: Aug. 7, 2024

Related U.S. Application Data
(60) Provisional application No. 63/308,283, filed on Feb. 9, 2022.

(51) Int. Cl. B05B 7/24; B05B 5/043; B05B 7/08; B05B 7/10; B05B 12/04; B05B 12/06; B05B 12/14; B05B 15/00

(52) U.S. Cl. CPC B05B 7/2472; B05B 5/043; B05B 7/08; B05B 7/10; B05B 7/2416; B05B 7/2424; B05B 12/04; B05B 12/06; B05B 12/1472; B05B 15/00

(57) ABSTRACT

A dual-liquid spray apparatus for dispensing concurrently two different liquids, such as droplets, having a spray nozzle assembly with two nozzles, each having a nozzle axis for dispersing a spray pattern of a liquid along the nozzle axis. In some embodiments, the dual-liquid spray apparatus can dispense the two liquids concurrently and simultaneously, where the nozzle axis of the first nozzle diverges at a diverging angle from the nozzle axis of the second nozzle. In some embodiments, the dual-liquid spray apparatus can dispense the two liquids concurrently and alternatingly, where a first supply means provides a series of pulses of the first liquid composition to the first nozzle, and a second supply means provides a series of pulses of the second liquid composition to the second nozzle alternatingly and non-overlapping with the pulses of the first liquid composition.

### FIELD OF THE INVENTION

This invention is related to the field of dispersing liquid, typically aqueous, compositions into rooms or other volumetric spaces.

### BACKGROUND OF THE INVENTION

Often, there is a need to disperse two or more liquid or aqueous compositions into a room or other volumetric space, for distribution and depositing onto one or more surfaces within. The compositions can either be dispersed sequentially, to form one or more layers of material on the surface(s), or simultaneously, where the compositions can interact with each other within the airspace and/or on the surface(s). In some instances, it can be advantageous to delay or prevent the liquid compositions or droplets thereof from contacting each other in the airspace, prior to depositing onto a surface, for example, to comply with regulatory requirements, promote safety, enhance composition stability, avoid a chemical reaction between a first reactive components in one liquid composition with a second reactive component in a second liquid composition, and/or optimize the benefits of the combined liquid compositions on the surface(s).

As a result, there is a need to develop an improved method and system for distributing two or more different compositions from a single device while delaying or preventing the liquid compositions or droplets thereof from interacting or coalescing with each other, in an airspace and in flight within a volumetric space, prior to the depositing of the two liquid compositions onto a surface, and coalescing the two or more different compositions into a combined layer of the two or more different compositions.

### SUMMARY OF THE INVENTION

The present invention relates to a system(s), device(s) and method(s) for the concurrent dispersion of two or more liquid compositions, into and through an airspace, while reducing, minimizing, or eliminating the mixing or coalescing together of the two or more liquid compositions within the airspace.

### Dispersed Droplet and Stream Forms

The dispersion can be a multiplicity of droplets, a stream or streams, or a combination thereof. The dispersions of the two or more liquid compositions deposit independently onto a surface or plurality of surfaces. The dispersions can be aimed and directed at the surface or surfaces, or can be aimed and directed into the airspace of volumetric space, for example a room, or enclosed or open area, to travel through the airspace and deposit under gravity onto a surface.

The present invention provides for a single device or multi-device system for, and a method for, dispersing the two or more liquid compositions, into the streams or droplets. The apparatus and method for dispersing the two or more liquid compositions are configured to reduce, minimize, or eliminate the mixing or coalescing together the respective droplets and streams of the two or more liquid compositions within the airspace, prior to their respective depositing onto the surface or surfaces.

The present invention also provides a spray nozzle assembly including two or more separate nozzles, as described herein.

The two or more liquid compositions can be different or distinct liquid compositions. In various embodiments, the first and second liquid compositions are dispersed independently and separately as a multiplicity of droplets.

A dispensing of droplets of the liquid composition from a spray nozzle can be propelled by the pumping rate or pressure applied to the liquid within the spray nozzle assembly, or by a volumetric flow of a pressurized air stream passing concurrently through the spray nozzle assembly in a two-fluid (liquid and air) spray nozzle assembly. The pressurized air stream can be generated by an air pump or a high-pressure air blower, for example a Hurricane fogger available from B&G Equipment Company. A stream of dispensed liquid composition from a spray nozzle can be propelled by the pumping rate or pressure applied to the liquid within the spray nozzle assembly.

An interaction of the dispersed droplets of the first liquid composition from the first liquid nozzle with the dispersed droplets of the second liquid composition from the second liquid nozzle is reduced by: 1) increasing the distance of the first nozzle and its dispersion vector (or nozzle axis), from the second nozzle and its dispersion vector (or nozzle axis), 2) increasing the relative dispersing angle between the nozzle axis of the first nozzle and the nozzle axis of the second nozzle, 3) reducing, minimizing, or preferably avoiding, dispersing of the droplets of the second liquid compositions while the droplets of the first liquid compositions are being dispersed, and visa versa, and 4) adding the same electrostatic charge to each of the droplet dispersions of the liquid compositions.

### Separation Distance of Two Nozzles

In various embodiments, the distance between the first and second nozzles is at least 1 cm, or at least 2 cm, or at least 4 cm, and up to about 10 cm, or up to about 6 cm. The distance should be sufficient to minimize or avoid overlapping and impingement of the dispersion of the first liquid composition with the second liquid composition when dispersed simultaneously.

### Reactant Compounds

In various embodiments, the two or more liquid compositions each can comprise a respective and different reactant compound, and when the two or more liquid compositions are combined into a mixed composition, the two different reactant compounds can react with one another to form a reaction product within the mixed composition. For example, a first liquid composition can comprise, consist of, or contain a first reactant compound, and a second liquid composition can comprise, consist of, or contain a second reactant compound. Upon combining the first liquid composition with the second liquid composition to form a resulting mixed, liquid composition, the first reactant compound and the second reactant compound react to form the reaction product.

### Catalyzed Reaction

In alternative embodiments employing a method, device or system described herein, a first liquid composition can comprise two or more reactant compounds that are stable, minimally reactive or non-reactive within the first liquid composition, while a second liquid composition can comprise a catalyst material that can trigger or initiate a reaction, or increase a rate of a reaction, among the two or more reactant compounds to form a target product.

### Concurrent Dispersing

The two or more liquid compositions are dispersed concurrently, wherein each of the two or more liquid compositions are dispersed within a concurrent period of time, or within each of a series of concurrent periods of time. During the concurrent period of time, each of the two or more liquid compositions are dispersed, either alone or together with the other liquid composition. For example, the first liquid composition is dispersed alone or only at a moment during the concurrent period of time, or is dispersed simultaneously with the second liquid composition at a moment during the concurrent period of time, and the second liquid composition is dispersed alone or only at a moment during the concurrent period of time, or is dispersed simultaneously with the first liquid composition at a moment during the concurrent period of time.

Without being bound by any particular theory of operation or function, the concurrent dispersing of the first and second liquid compositions into a volumetric space improve the reliability and uniformity of the volume or mass of the first and second liquid compositions, and their relative volumes and/or masses, deposited and coalesced onto the one or more target surfaces. Typically each portion, region or area of the one or more target surfaces is covered by a reaction liquid layer comprising a reaction product, which is formed by the first reactant compound of the first liquid composition, and the second reactant compound of the second liquid composition, where the first and second reactant compounds and/or the reaction product are present within each portion, region or area of the one or more target surfaces, in an amount or concentration that is similar, the same, or uniform, or within a predetermined or acceptable range.

### Concurrent, Simultaneous and Divergent Dispersing

In various embodiments of a system or method, the two or more liquid compositions are dispersed concurrently and simultaneously into the airspace within a concurrent period of time, or within each of a series of concurrent periods of time, and are dispersed along divergent spray vectors. During the concurrent period of time, both of the two or more liquid compositions are dispersed together. For example, the first liquid composition is dispersed simultaneously with the second liquid composition at a moment during the concurrent period of time, and the second liquid composition is dispersed simultaneously with the first liquid composition at a moment during the concurrent period of time. During the concurrent period of time, in addition to the two or more liquid compositions being dispersed together, either of the first liquid composition or the second liquid composition can also be dispersed independent of the other. For example, the time during dispersing of the first liquid composition overlaps with time during the dispensing of the first liquid composition, and before or after such time either the first liquid composition only or the second liquid composition only may be dispersed.

The present invention provides a dual-liquid spray apparatus. for dispensing two different liquids. preferably as droplets. concurrently and simultaneously. comprising a spray nozzle assembly comprising at least two nozzles, each nozzle having a nozzle axis for dispersing a spray pattern of a liquid along the nozzle axis, wherein the nozzle axis of a first nozzle for dispersing a first liquid composition diverges at a diverging angle from. and is not parallel to. the nozzle axis of a second nozzle for dispersing a second liquid composition, a first means for supplying the first liquid composition to the first nozzle, and a second means for supplying the second liquid composition to the second nozzle.

The present invention also provides a spray nozzle assembly comprising at least two nozzles, each nozzle having a nozzle axis for dispersing a spray pattern of a liquid along the nozzle axis, wherein the nozzle axis of the first nozzle for dispersing a first liquid composition diverges at a diverging angle from, and is not parallel to, the nozzle axis of the second nozzle for dispersing a second liquid composition.

In various embodiments, the diverging angle is at least 3 degrees, and preferably at least 10 degrees.

In the various embodiments, during the concurrent and simultaneous dispersing of the two or more liquid compositions, each dispersing comprises an ejecting or spraying of the respective liquid composition along a dispersing vector, and the dispersing vector of the first liquid composition diverges from, and is not parallel to, the dispersing vector of the second liquid compositions. In embodiments, or spraying of the respective liquid composition comprises or consists essentially of dispersing of droplets of the liquid composition.

In various embodiments, the dispersing source is a spray or droplet nozzle, including a first nozzle for the first liquid composition, and a second nozzle for the second liquid composition. The first nozzle and the second nozzle can be disposed within a single dispersing apparatus or within separate apparatuses. The first nozzle and the second nozzle can be oriented within a virtual plane and separated by a distance. In some embodiments, the first nozzle and the second nozzle are oriented in a substantially horizontal plane, substantially perpendicular to the force of gravity, the first nozzle has a nozzle axis that diverges from, and is not parallel to, a nozzle axis of the second nozzle. The nozzle axis of the first nozzles diverges from the nozzle axis of the second nozzle by a diverging angle a, and the first and second dispersing vectors of the dispersions of the first and second liquid composition diverge at a diverging angle roughly the same as the diverging angle a of the nozzle axes. In various embodiments, the diverging angle a between the first and second nozzle axes can be up to 45°, and can be at least 3°, more typically at least 6°, or at least 10°, and up to about 30° or more, though more typically up to about 25°, or about 20°.

At any moment during dispersing, the dispersing vector of the first liquid composition is non-parallel to, and diverges outwardly and away from, the dispersing vector of the second liquid composition. In various embodiments, the dispersing vectors of the droplets of the two liquid compositions follow along the respective nozzle axes of the spray nozzles to a target distance, T, with the droplets dispersing axially along, and divergently away from, the dispersing vectors, as shown in FIG. 8. As the dispersed droplets move axially along the dispersing vectors, they migrate radially away from the dispersing vector, typically in a circular or elliptical pattern in a cross-section normal to the dispersing vector. For example, the droplets expand in a conical pattern, centered angle along the dispersing vector, and having a dispersing angle, denoted angle β, centered along the respect nozzle axis. The dispersing angle β of the dispersing droplets can depend upon several factors, including the compositional and electrostatic properties of the droplets of the liquid composition, the design of the spray nozzle, and the operation of the droplet propulsion device of the spraying apparatus. Typically, the aqueous liquid droplets disperse in a pattern having a resulting dispersing angle β of about 10° to about 30° in the horizontal plane.

Without being bound by any particular theory of operation or function, the angling away of the dispersions of the first and second liquid compositions directs the momentum of the respective droplets of the first and second liquid compositions away from one another, particularly within the first several, such as 3-6 feet (1-2 meters) from the spray nozzles. As the droplets continue along the spray vectors, they begin to slow in velocity and momentum, and the pattern of droplets can begin to expand slightly outwardly, away from the dispersing vector, at further distances from the spray nozzles, and the interparticle distance increases, thereby reducing or minimizing collisions between droplets, and in particular between a droplet of the first liquid composition with a droplet of the second liquid composition.

As illustrated in FIG. 8, the diverging angle a of the nozzle axes is configured and selected to limit or prevent the radially-diverging dispersing pattern of the first liquid composition 51 from overlapping with the radially-diverging conical dispersing pattern of the second liquid composition 52. Minimizing or eliminating the overlap of the dispersing patterns of the first and second liquid compositions minimizes or eliminates contact of droplets of the first liquid composition with droplets of the second liquid composition, and formation of droplets of the mixed composition.

In various embodiments of the invention, the first liquid composition and the second liquid composition each comprise at least one reactant compound and/or a reactant catalyst. In particular embodiments of the invention, the first liquid composition comprises hydrogen peroxide or a peroxide-producing compound, and the second liquid composition comprises an organic acid, and preferably acetic acid, and the formation of droplets of the mixed composition results in situ in the formation of peracid, and preferably a peracetic acid. The formation of particles or droplets of a mixed composition comprising a peracid, such as peracetic acid, raise a risk of inhalation of droplets comprising peracid, or peracetic acid, which can be harmful to humans and mammals.

Without being bound by any particular theory, a diverging angle a of the two nozzle axes is selected to optimize the performance of the two-liquid spraying system, while minimizing the mixing and coalescing of the droplets of the two difference liquid compositions. The latter is important when the two liquid compositions consist of two different liquid compositions, each containing a different reactant compound, which only form a reactant product when the two different liquid compositions combine form a mixed liquid composition, and the two different reactant compounds react and form the reaction product in the mixed liquid composition.

When the two different liquid compositions are being dispersed as respective patterns of droplets, increasing the diverging angle a of the two nozzle axes reduces the interaction and coalescing of the droplets from the two different droplet patterns, as the diverging angle increases, until interaction and coalescing of different droplets is negligible, unmeasurable or avoided.

On the other hand, to provide an effective mixture of the coalesced droplets onto a surface or surfaces sufficient to form an efficacious reaction product, it is important for sufficient quantities of droplets of both of the two different liquid compositions to travel through the airspace, and to descend, land on, and coalesce on a surface, to form a coalesced mixed composition contained the reactant product. When the two different liquid compositions are being dispersed as respective patterns of droplets, reducing or minimizing the diverging angle a of the two nozzle axes increases the delivery and coalescing of sufficient amounts of droplets from both different liquid composition to provide effective performance on all surfaces toward which the dispersing spray patterns were directed. For example, in the case of two reactant compounds that form a sanitizing reactant product, it is important to deliver sufficient (for example, stoichiometric) quantities of both reactant compounds onto the surface or surfaces, to form an effective sanitizing product that can sanitize such surfaces.

### Concurrent, Alternatingly Dispersing

In various embodiments, the first liquid composition and the second liquid composition are dispersed concurrently and alternatingly into the airspace within a concurrent period of time, or within each of a series of concurrent periods of time. During the concurrent period of time, both of the two or more liquid compositions are dispersed separately and independently from one another, at mutually exclusive times. The first liquid composition is dispersed in a series of pulses, each in a volumetric pulse amount during a pulse time, each pulse of the series of pulses being separated in time by a dwell time. The second liquid composition is also dispersed in a series of pulses, each in a volumetric pulse amount during a pulse time, each pulse of the series of pulses being separated in time by a dwell time. The pulses of the first liquid composition are dispersed during the dwell time of the pulses of the second liquid composition, while the pulses of the second liquid composition are dispersed during the dwell time of the pulses of the first liquid composition, and do not overlap in time with the pulses of the first liquid composition.

In various embodiments in which a liquid composition dispersion includes a multiplicity of droplets, the dispersed pulse of the liquid composition comprises a multiplicity of droplets that travel in a wave that diverges radially away from the dispersing vector as the pulse moves axially along the dispersing vector. Typically the pulsed wave of droplets diverges in a circular or elliptical pattern in a cross-section normal to the dispersing vector. For example, the droplets of each wave expand in a conical pattern, substantially at a radially dispersing angle along the dispersing vector. For example, the droplets expand in a conical pattern having a dispersing angle, denoted angle β. The dispersing angle β can be about 10° to about 40°.

In various embodiments, the first and second liquid compositions 51,52 are dispersed in pulses (pᵢ, FIG. 9) from two separate nozzles. The dispersing vectors of the first and second liquid compositions can be parallel and spaced apart by a distance sufficient to accommodate the two nozzles. In alternate embodiments, the first and second liquid compositions are dispersed in pulses from a single nozzle, along a single dispersing vector. The series of alternating pulse dispersions of the first and second liquid compositions move in axially spaced apart pulse waves along the respective dispersing vectors or single vector. The dwells of the alternating pulse dispersions are sufficient to separate the alternating pulse dispersions of the first and second liquid compositions in space and time to minimize or eliminate overlap of the alternating dispersing pulses of respective droplets of the first and second liquid compositions, to minimize or eliminate contact of the droplets of the first liquid composition with the droplet of the second liquid composition, and formation of droplets of the mixed composition.

Without being bound by any particular theory of operation or function, the pulse waves of droplets of the first liquid composition and the pulse waves of droplets of the second liquid composition distribution through the airspace without overlapping and intermixing, or at least without significant overlapping. Typically, only the most energetic (or fastest-moving) droplets from a trailing pulse wave of the first liquid composition (or second liquid composition) are able to overtake the least energetic (or slowest-moving) droplets of the leading pulse wave of the second liquid composition (or first liquid composition).

In various embodiments, the duration of a dispersion of a pulse wave of either a first liquid composition or a second liquid composition can be a period of time of at least 0.01seconds to 10 seconds. In other embodiments, the duration of a dispersed wave or pulse is at least 0.1 seconds, or at least 0.25 seconds, and up to about 1 second, or up to about 0.5 seconds, or up to about 0.25 seconds. Each pulse wave of droplets of the first liquid composition has a wave or pulse distance (ym, FIG. 9) from the leading edge to the trailing edge of the wave, and each pulse wave of droplets of the second liquid composition has a wave or pulse distance (yn),. Each pulse of the first liquid composition is separated by a dwell time that creates a dwell distance (wm, FIG. 9), and each pulse of the second liquid composition is separated by a dwell time that creates a dwell distance (wn). In various embodiments, the dwell of the first and second liquid composition dispersions effect a time delay between, and a separation distance (z, FIG. 9) between the ends of pulse waves of droplets of the first liquid composition and the beginnings of pulse waves of the droplets of the second liquid composition, and visa versa. In various embodiments, the time delay is at least 0.01 seconds and up to 1 second. In other embodiments, the time delay is at least 0.1 seconds, or at least 0.5 seconds, and up to about 0.5 second, or up to about 0.25 seconds, or up to about 0.1 seconds. The distance delay between the ends and beginnings of the pulse waves of droplets of the first liquid composition and the droplets of the second liquid composition is sufficient to minimize or eliminate contact and mixing of the droplets of the first liquid composition with the droplet of the second liquid composition, and formation of droplets of a mixed composition.

### Concurrent, Alternatingly and Divergent Dispersing

In various embodiments, the first liquid composition and the second liquid composition are dispersed concurrently and alternatingly and along divergent spray vectors.

FIG. 10 illustrates the concurrent dispersing of droplets of the first and second liquid compositions 51,52 along divergent vectors centered on the diverging axes of the two nozzle 10,20. Each of the first and second liquid compositions 51,52 is dispersed in pulses or waves in alternative dispersing periods, during the dwell of the other. In this embodiment, both mechanisms effect the reducing, minimizing, or preventing of the mixing of droplets of the first liquid composition with the first liquid composition within the airspace.

### Droplet Formation

In various embodiments, a multiplicity of droplets of a liquid composition can be formed as selected from the group consisting of a coarse spray, a mist, a shower, an aerosol, a fog, and a vapor, and combinations thereof, by any one or more well-known methods. In various embodiments, at least about 90 percent, which can be at least 95 percent, or at least 97 percent, or at least 98 percent, or 99 percent, by volume of first and second liquid compositions are dispersed as a multiplicity of droplets. In further embodiments, essentially 100 percent by volume of either of or both of the first and second liquid compositions are dispersed as a multiplicity of droplets. Sprayers, fogging machines, and other devices for dispersing the first and second liquid compositions as droplets are well-known in the art.

### Aqueous Liquid Compositions

In various embodiments, one or more of the liquid compositions is an aqueous composition comprising water as a solvent. In various embodiments, one or more of the liquid compositions can be non-aqueous compositions, including but not limited to oil-based compositions, organic compounds or compositions, and other volatile compounds or compositions that are substantially free of water. In various embodiments, all of the liquid compositions that are dispersed are aqueous compositions.

### Diameter Size

In various embodiments, the equivalent diameter of the multiplicity of droplets can be controlled to be small enough to enable the droplets to distribute the airspace and throughout a portion of the volumetric space. In various embodiments, the formation of droplets of the liquid compositions can be controlled so that at least 90%, typically at least 95%, and even more typically at least 99% of the droplets, by volume of the dispersed liquid composition, have a diameter (or equivalent diameter) within a desired or target droplet-size range. For the formation of droplets (smaller droplets) that can be carried through the airspace and remain suspended in the airspace for a distance of about 10 feet (about 3.3 meters) or more, the target droplet-size range is at least 10 microns, or at least 15 microns, and up to 100 microns; preferably up to 80 microns, or up to 50 microns, or up to 25 microns. For the formation of droplets (medium droplets) that can be carried through the airspace but descend by gravity within a distance of about 15 feet (about 5meters) or less, the target droplet-size range is at least 25 microns, or at least 50 microns, and up to 250 microns; preferably up to 125 microns, or up to 100 microns, or up to 75 microns.

In various embodiments, the dispersion and distribution of one or more aqueous compositions can be controlled to increase, enhance, or maximize the combining of the compositions within the volumetric space, prior to depositing onto target surfaces. In various embodiment, the dispersion and distribution of one or more aqueous compositions can be controlled to reduce, minimize, or prevent the combining of the compositions within the volumetric space, prior to depositing onto target surfaces.

### Multi-Sprayer Dispersion

In various embodiments, the two or more liquid or aqueous compositions can be dispersed using a system comprising multiple spray devices situated at different locations within the volumetric space. In various embodiments, the two or more liquid or aqueous compositions can be dispersed as a multiplicity of droplets using a system comprising a single spray device.

### Multi-Nozzle Dispersion

In further embodiments, within a single spray-device system, the spray device can comprise: (a) two or more liquid or aqueous composition containers, each container configured for housing or containing a separate and different liquid or aqueous composition; (b) two or more means for pumping or drawing a liquid or aqueous composition from one of the two or more of the liquid or aqueous composition containers; and (c) two or more delivery nozzles, each delivery nozzle in fluid communication with at least one of the means for pumping and configured to dispense a volumetric or mass quantity of the liquid or aqueous composition. In various embodiments, the two or more delivery nozzles of the single spray-device system disperse the respective liquid or aqueous composition into a multiplicity of droplets, or a size or diameter described herein above. In various embodiments, the axial velocity of the dispersed droplets or pulse waves of the first liquid composition is substantially the same as the axial velocity of the dispersed droplets or pulse waves of the second liquid composition.

In various embodiments, the above spraying systems can be configured to disperse the two (or more) liquid or aqueous compositions concurrently, each of a predetermined mass or volumetric rate, and each emitted at a corresponding axial velocity depending upon the design of the nozzle, and the axial mass or volumetric airflow rate through the two-fluid spray nozzle, and the volumetric rate or pressure of the respective liquid composition. In a non-limiting example, a first liquid or aqueous composition is dispersed into a volumetric space, concurrently (and either or both alternatingly or divergently) with a second liquid or aqueous composition being dispersed into the volumetric space.

In various embodiments, the total amount of time for the concurrent dispersion of the first and second liquid compositions can be determined by calculation based on the square footage of surfaces to be contacted, for example, within the volumetric space) and the determined or target equivalent thickness of the coalesced reaction layer upon the surface(s). In a non-limiting example, for a typical facility, office or residence room, the concurrent dispersing of the two liquid compositions onto the surfaces within the room takes about 1 to 15 minutes.

### Airspaces

Non-limiting examples of volumetric spaces having an airspace can include rooms, buildings, shipping containers, and transportation compartments, whereas non-limiting examples of target surfaces are food-processing surfaces, health care surface and instruments, laboratory surfaces, industrial or commercial machinery, or surfaces in homes and residences. The potential applications are diverse. One non-limiting example of such an application is the disinfecting of surfaces using two or more aqueous compositions. Such methods, systems, and devices are described in U.S. Pat. Nos. 9,861,102 and 10,603,396; U.S. Patent Publication Nos. 2019/0091360 and 2020/0306399; U.S. Patent Application No. 62/985,783 and PCT Publication Nos. WO 2017/205649, 2019/075176, and 2021/178774, the disclosures of which are incorporated by reference in their entireties.

### Nozzle Outlet Shapes

The shape of the design of the spray nozzle can affect the shape of pattern of dispersed droplets from the spray nozzle assembly. In most conventional spray nozzles, the shapes of the liquid supply and outlet tubes, and of the air supply, venturi and air outlet tubes, are circular in cross section along the nozzle axes, resulting in a dispersed pattern of droplets being emitted from the spray nozzle having a circular pattern in a cross section normal to the dispersing vector. Typically, the

In various embodiments, the shape of the liquid outlet tube, and the air venturi and air outlet tubes, are either rectangular or elliptical, oriented with the long axis in the vertical direction (direction of gravitational force during spraying use), which is turn results in a dispersed pattern of droplets being emitted from the spray nozzle having a circular pattern in a cross section normal to the dispersing vector. The narrowing of the dispersed pattern of droplets in the horizontal or lateral direction helps to reduce and minimize the overlapping of the respective dispersed patterns of the first and second liquid compositions.

### Spray Patterns

In various embodiments, a spray nozzle of a spray device disperses the liquid composition in a spray pattern. Non-limiting examples of a spray pattern can be a solid, circular or oval-shaped cone emitted along the nozzle axis, a hollow, circular or oval-shaped cone emitted along the nozzle axis, and a flat or planar spray emitted transverse the nozzle axis.

### ESS

In various embodiments, though preferred in the systems and methods of the present invention that generate droplets of the two liquid composition, a droplet of either one or both the first and second liquid compositions dispersed from a nozzle can be charged electrostatically. In some embodiments, the droplets of both the first and second liquid compositions dispersed from a nozzle can be charged electrostatically with the same electrostatic charge, which can be either a positive charge or a negative charge.

The electrostatic charge is usually applied to the dispersed droplets of the liquid composition as the exit the spray nozzles, using ionizing or high voltage charging system comprising pin electrodes and a charging (ground) ring, which pass the high voltage charge to the liquid droplets passing therebetween. Typical electrostatic charges on the dispersed droplets are between 5 to 20 kilovolts (kV), such as 8-10 kV.

Without being bound by any particular theory, use of an electrostatic charge of the dispersions of droplets of both the first and second liquid compositions causes individual droplets to repel electrostatically one another. When the two dispersed streams of droplets are emitted at high velocity and momentum from the spraying apparatus, collisions between droplets that might occur will involve droplets of the same liquid composition, so that reaction products are not formed. As the droplets proceed along their respective dispersing vectors, their velocity and momentum slow, and the distance between droplets in each dispersing patterns expand laterally, away from the dispersing vector, as the result of the repelling force of the electrostatic charge between droplets (the repulsive force between two droplets due to electrostatic charge is proportional to the square of the distance between the droplets). The collisions between and mixing of any two droplets, including droplets of the same liquid compositions and droplets of different liquid compositions, are reduced because of droplets' mutual electrostatic repulsion, until all the droplets eventually descend, land on, and coalesce on a surface.

### Axial Velocity and Kinetics

In various embodiments, the kinetic energy and axial velocity of the dispersed droplets of each of the first and second liquid compositions can be affected and controlled by an amount of power applied per unit of liquid dispersed. The amount of power applied by an air pump or a blower is believed to correlate with the airflow rate and air pressure generated within the housing, and the amount of the liquid solutions drawn by venturi action from the liquid container(s). The amount of power applied by a liquid pump sprayer is believed to correlate with the volumetric flow rate and liquid pressure generated within the housing. Without being bound by any particular theory of operation or function, an increase in the amount of power applied per unit of liquid dispersed can result in an increase in the volumetric output of the dispersed liquid composition(s), a decrease in droplet diameter or size, an increase in the time-of-flight and travel distance of the droplets, and an increase in the interaction of dispersed droplets, while a decrease in the amount of power applied per unit of liquid dispersed can result in an increase in droplet diameter or size, and a decrease in the time-of-flight of the droplets. Significantly increased droplet size and the decrease in time-of-flight can result in pre-mature coalescing of droplets and deposition of the dispersed liquid composition in the immediate area, such as on the floor, surrounding the spray device.

### Auxiliary Electronic Device

A dual- (or multi-) liquid droplet sprayer apparatus of the present invention can also include an auxiliary electronic device, such as an illuminating lamp or a directional signal, illustrated as element 31 in FIG. 1. The auxiliary electronic device be connected for power and control to the respective power and control units of the dual-liquid spray nozzle assembly. In one example, an auxiliary electronic device can be an ultraviolet light lamp, such a UV-light emitting diode (UV-LED) for illuminating the space in front of the dual-liquid spray nozzle assembly with UV light, for providing or assisting sanitizing operations using the dual-liquid spray nozzle assembly and apparatus. In another example, an auxiliary electronic device can be a laser lamp, for emitting a beam of visible or invisible laser light in front of the dual-liquid spray nozzle assembly, to providing a directional signal for the dual-liquid spray nozzle assembly and apparatus.

The invention is further illustrated by the following working and prophetic examples, neither of which should be construed as limiting the invention. Additionally, to the extent that section headings are used, they should not be construed as necessarily limiting. Any use of the past tense to describe an example otherwise indicated as constructive or prophetic is not intended to reflect that the constructive or prophetic example has actually been carried out.

### BRIEF DESCRIPTION OF THE FIGURES

FIG. 1 shows a dual-liquid droplet sprayer apparatus of the present invention.

FIG. 2 shows a front, right perspective view of a dual-liquid spray nozzle assembly having a pair of spray nozzles, contained within a housing body of the dual-liquid droplet sprayer apparatus.

FIG. 3 shows a rear, right perspective view of the dual-liquid spray nozzle assembly having a pair of liquid supply ports for the respective pair of spray nozzles.

FIG. 4 shows a front face view of the dual-liquid spray nozzle assembly.

FIG. 5 shows a top view of the dual-liquid spray nozzle assembly, showing the liquid supply ports aligned with their respective spray nozzles along diverging nozzle axes.

FIG. 6 shows a top-plan sectional view of the dual-liquid spray nozzle assembly, taken along the two diverging nozzle axes and line 6-6 of FIG. 4.

FIG. 7 shows a side-elevation sectional view of the dual-liquid spray nozzle assembly, taken along a first diverging nozzle axes and line 7-7 of FIG. 7.

FIG. 8 shows a top plan schematic view of the dual-liquid spray nozzle assembly illustrating the first liquid composition and the second liquid composition are dispersed concurrently and simultaneously, with diverging angling of the nozzle axes of the two spray nozzles, and the diverging dispersing patterns of the two dispersed liquid compositions centered along the divergently-angled nozzle axes.

FIG. 9 shows a top plan schematic view of a second embodiment of a dual-liquid spray nozzle assembly illustrating the first liquid composition and the second liquid composition are dispersed concurrently and alternatingly.

FIG. 10 shows a top plan schematic view of a third embodiment of a dual-liquid spray nozzle assembly illustrating the first liquid composition and the second liquid composition are dispersed concurrently and alternatingly, with diverging angling of the nozzle axes of the two spray nozzles, and the diverging dispersing and alternating patterns of the two dispersed liquid compositions centered along the divergently-angled nozzle axes.

### DETAILED DESCRIPTION OF THE INVENTION

### Definitions

As used in this specification and in the claims, the singular forms “a,” “an,” and “the” include plural referents unless the content clearly dictates otherwise.

The term “about” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Similarly, whether or not a claim is modified by the term, “about,” the claims included equivalents to the quantities recited.

As used herein, the term “aqueous composition” refers to a combination of liquid components that includes water. Most commonly, aqueous compositions are synonymous with the term “solution” as it is commonly used in the art for this invention. However, depending on the identity of components in the composition in addition to water, “aqueous compositions” can also encompass mixtures, emulsions, dispersions, suspensions or the like. Furthermore, while water must be present, it need not comprise the majority of the aqueous composition.

As used herein, the term, “concurrent” means the two or more liquid compositions are both dispersed independently within a period of time, or within each of a series of periods of time.

As used herein, an “equivalent diameter” of a non-spherically-shaped droplet refers to the diameter of a sphere of a liquid that is equivalent in mass to the non-spherical-shaped droplet of the liquid, such as where gravity causes a droplet to form a “teardrop” or “raindrop” shape.

As used herein, the terms “free” or “substantially free” refer to the total absence or near total absence of a particular compound in a composition, mixture, or ingredient.

As used herein, the term “reaction layer” refers to a layer formed on a surface arising from the combining or coalescing of at least a first aqueous composition and a second aqueous composition upon the surface. Each aqueous composition can comprise one or more compounds, whereupon the formation of a reaction layer, react with each other within the reaction layer to form a product in situ.

As used herein, the term, “volumetric space” refers to atmosphere or the air space within a room or other contained space, typically on fixed and determinable volume, and typically containing objects or structure therein having surfaces.

In describing embodiments of the methods and systems in the present disclosure, reference will be made to “first” or “second” as they refer to aqueous compositions. Except when there is clear context that a specific order is intended, “first” and “second” are merely relative terms, and a “first” composition described could just as easily and conveniently be referred to as a “second” composition, and such description is implicitly included herein.

### Embodiments of the Invention

### In Situ Peracid Formation

In one embodiment of the invention, a first liquid composition comprises an aqueous composition with a first reactant compound comprising hydrogen peroxide, and a second liquid composition comprises an aqueous composition with a second reactant compound comprising an organic acid. The first liquid composition comprises at least 0.1% and up to 15% hydrogen peroxide, and the second liquid composition comprises at least 0.1% and up to 20% an organic acid. The two liquid compositions are dispersed simultaneously as droplets into a volumetric space (airspace). Either or both of the first liquid composition and the first liquid composition can also comprise independently from at least 0.1% and up to 25% an alcohol, which can be one or more alcohols of the following: ethanol, propanol, isopropanol, butanol and its isomeric structures, pentanol and its isomeric structures, and hexanol and its isomeric structures.

Non-limiting examples for first and liquid compositions comprising respective first and second peracid reactant compounds, such as hydrogen peroxide and organic acid, can be found in US Patent Publications 2020/0306399 and 2021/0015954, and International Patent Publication WO 2021/178774, the disclosures of which are incorporated by reference in their entireties.

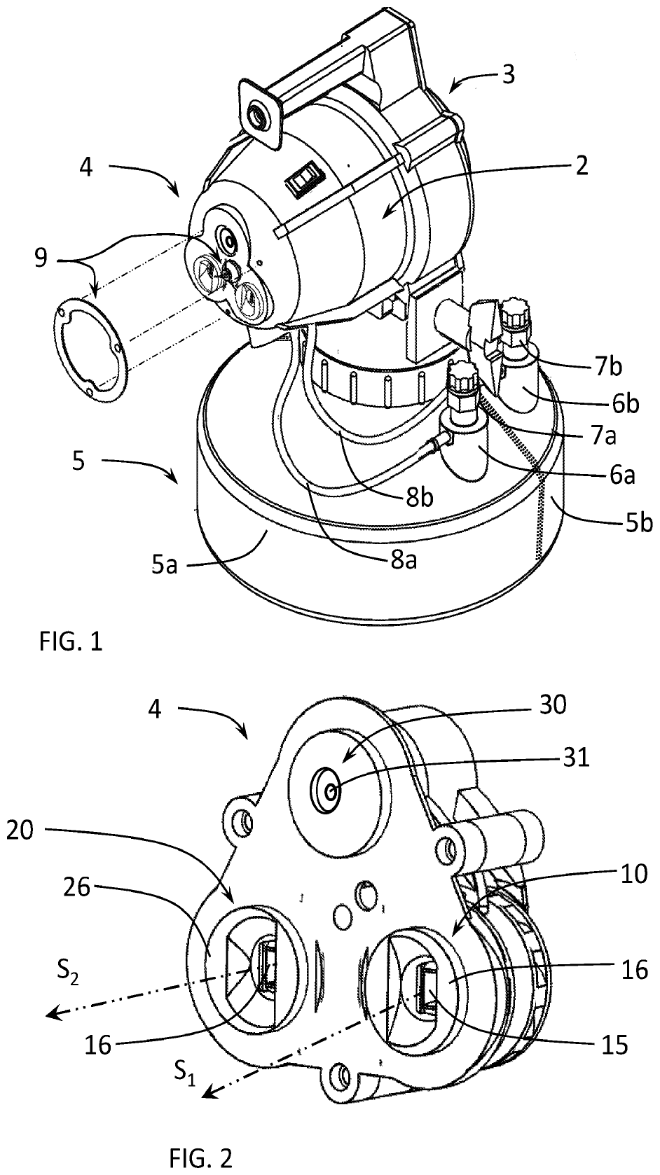

Each of the first and second liquid compositions can be contained within and dispensed by a multi-nozzle liquid dispensing device illustrated in FIG. 1. The multi-nozzle liquid dispensing device includes a blower assembly, a nozzle system comprising at least two nozzles including a first nozzle and a second nozzle formed within a nozzle body, a housing for the blower and the nozzle body, at least two separate formulation tanks, including a first tank and a second tank, and at least one or more metering valves in fluid communication with and between the first tank and the first nozzle, and the second tank and the second nozzle. The blower can be a two-stage axial compressor driven by a universal motor operating at a speed of up to 20,000 RPM. The blower moves a large mass and volume of air through the nozzles of the nozzle system. A volumetric flow of both the air and respective liquid compositions are drawn through each of the two nozzles, with the respective liquid compositions dispersed into tiny aerosol droplets and dispersed into the atmosphere. The flow rate of the first and second liquid compositions can be controlled manually or remotely with the metering valve. Each of the first and second liquid compositions is contained within the separate and respective first and second tanks. Generally, the sizes of the output droplets increase with increasing liquid flow rate and with increasing viscosity of the liquid being dispensed.

Other means of imparting energy to dispense a stream or a dispersion of droplets of a liquid composition can include pumps, centrifugal misters, and piezo-electric misters.

In various embodiments, a conventional multi-nozzle liquid dispensing device can be modified to form the multi-nozzle liquid dispensing device of the present invention. A modification of or change from a conventional multi-nozzle liquid dispensing device can include the addition of a second formulation tank, or the replacement of the conventional formulation tank with at least two separate formulation tanks. A modification of or change from a conventional multi-nozzle liquid dispensing device can include the modification or the replacement of the conventional nozzle body to provide a nozzle body having at least two nozzles that have separate and respective fluid communication with first nozzle and second nozzle of the present invention through separate liquid supply lines. with at least two separate formulation tanks. A conventional nozzle body has two or more nozzles that are in fluid communication to a single common liquid supply line to the single formulation tank. Non-limiting examples of a conventional nozzle body including spraying and fogging apparatus available from B&R Equipment Company under the brand names Dyna-Fog and Hurricane.

The first and second nozzles are disposed within the nozzle body with the outlets of the nozzles positioned along respective axes. The first and second nozzles outlets are spaced apart by a horizontal distance of about 1-3 inches (2.5-7.5 cm), though larger spacing can be used.

### WORKING EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. Thus, while the present invention has been described above with particularity, the following examples provide further detail in connection with what are presently deemed to be the most practical and preferred embodiments of the invention.

### Example 1: Dual Liquid Spraying

A single-liquid spray nozzle assembly from a Hurricane ES hand-carry electrostatic ULV fogger, available from B&G Equipment Company, was modified into a dual-liquid spray nozzle assembly. The conventional nozzle assembly has three spray nozzles arranged in a triangular pattern in the forward face of the nozzle assembly. Each spray nozzle includes a supply port that feeds liquid to an annular manifold, and the annular manifold has three outlets each in liquid communication with one of three venturi nozzle ports of the three spray nozzles. The conventional nozzle assembly also has separate air swirl channels, each channeling air from the blower, through the venturi passage and out through the spray nozzles to disperse the liquid droplets outward and along the dispersing vector.

The modified dual-liquid spray nozzle assembly can be assembled into a dual-liquid droplet sprayer apparatus as shown in FIG. 1. The sprayer apparatus 1 includes a housing body 2 for housing a blower 3 and a nozzle assembly 4, and has a housing base for connecting a cylindrical container 5. The cylindrical container 5 consists of a pair of semi-cylindrical-shaped containers 5a,5b assembled and secured together, and having a common upper connection for connecting to the base of the housing 2. Each of the first container 5a and the second container 5b has respective outlet ports 6a,6b, a pair of flow control valves 7a, 7b to control liquid flow from the respective containers 5a,5b, and liquid supply tubes 8a, 8b connecting the outlets of the flow control valves 7a, 7b to respective supply ports 11,21 of the modified nozzle assembly 4. The spray nozzle assembly also includes an ionizing device 9, illustrated in FIG. 1 as an ionizing or high voltage charging system comprising pin electrodes and a charging (ground) ring, proximate to the nozzles 10 and 20, for electrostatically charging the microdroplets of the aqueous composition dispensed by the nozzles. Typical electrostatic charges on the dispersed droplets are between 5 to 20 kilovolts (kV), such as 8-10 kV.

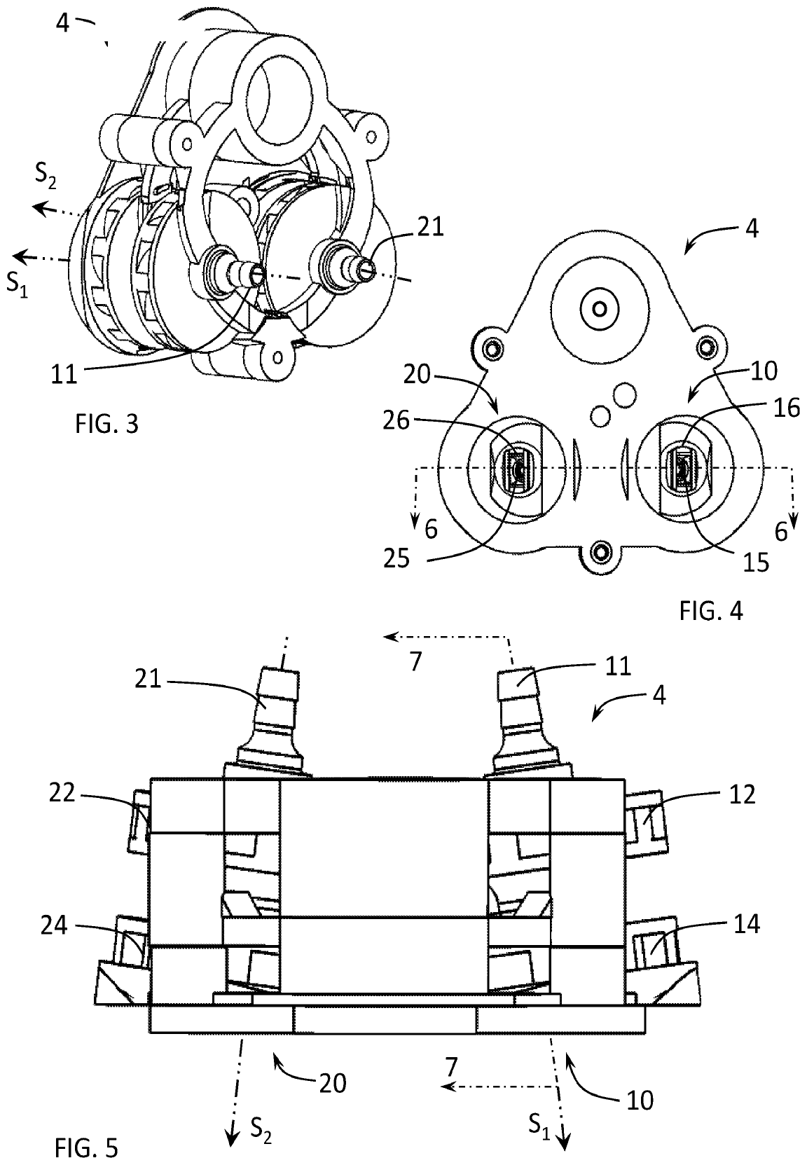

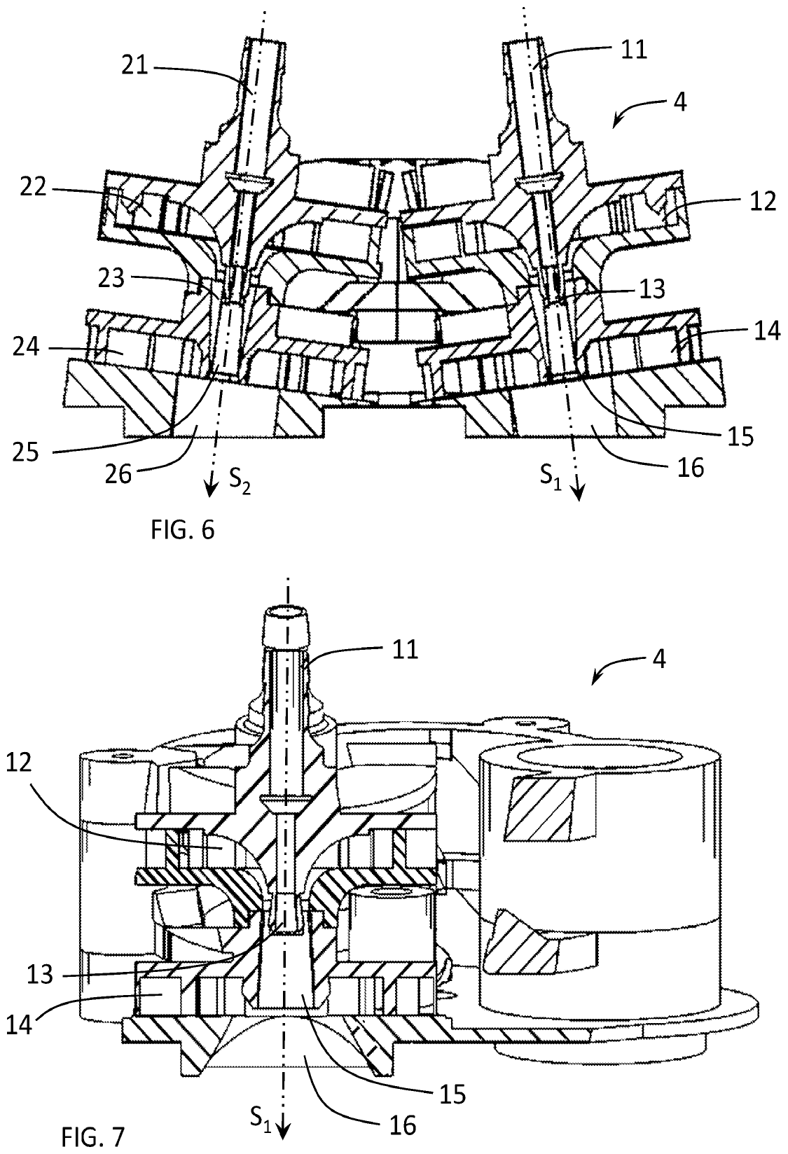

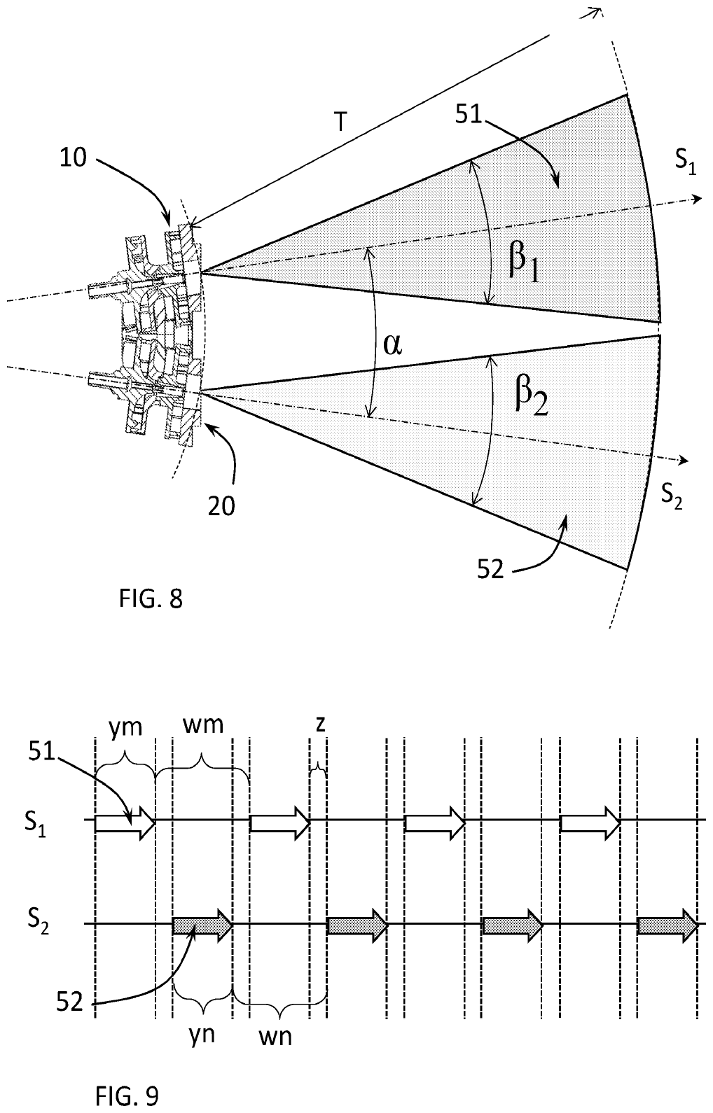

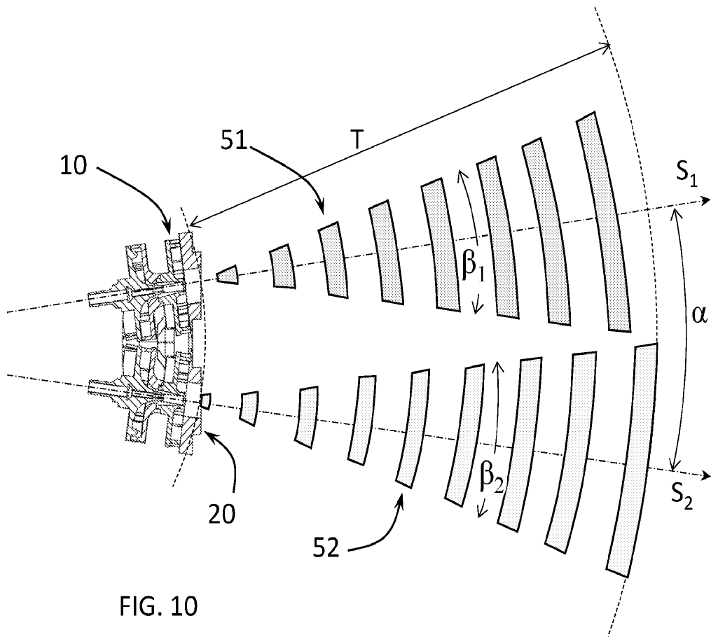

The modified dual-liquid spray nozzle assembly 4 is illustrated in FIGS. 2 through 7, and had an overall size and shape for mounting within a same housing body 2. The modified dual-liquid spray nozzle assembly 4 was made by additive manufacturing of a dual-liquid spray nozzle assembly model that was based upon a model of the conventional single-liquid spray nozzle assembly. The liquid supply port and manifold of the conventional nozzle assembly are replaced or modified to provide a separate, dedicated supply ports 11,21 to feed liquid from the flow control valves 7a, 7b to the respective spray nozzles 10,20. One of the three spray nozzles (the nozzle shown at the 12-o'clock position in a front view) of the conventional model was deleted or blocked off, and/or replaced with a blank position 30.

The remaining two spray nozzles can be left unchanged, to provide a pair of conventional spray nozzles having circular-shaped liquid supply and outlet tubes, and air supply, venturi and air outlet tubes, within the two nozzle axes oriented in parallel.

In various preferred embodiments, the remaining two spray nozzles 10,20, are modified to give rectangular shapes to the liquid outlet tube 15,25, and the air venturi 13,23 and air outlet ports 16,26, oriented with the long axis in the vertical direction (direction of gravitational force during spraying use), as shown in FIG. 4, and to cant or angle the nozzle axes S₁, S₂ at a diverging angle, as shown in FIG. 5.

The modified nozzle assembly 4 maintains the separate internal air swirl channels, including inner air swirl channels 12,22 that deliver a portion of air flow to the respective venturi 18,28, and outer air swirl channels 14,24 that deliver a portion of air flow to the nozzle outlet ports 16,26.

The centerline axis Si of the first spray nozzle 10 extends through the supply port 11, venturi tube 13, and air/liquid discharge port 15, and defines the dispersion vector for the dispersed first liquid composition, and the centerline axis S₂ of the second spray nozzle 20 extends through the supply port 21, venturi tube 23, and air/liquid discharge port 25, and defines the dispersion vector for the dispersed second liquid composition. In the illustrated embodiment of FIG. 5, the first and second spray nozzles 10,20 are configured to define a relative angle between nozzle centerline axis S₁ and nozzle centerline axis S₂ of about 15°.

First and second spray liquid compositions were supplied, and placed into the first and second containers 5a,5b. At a reduced setting of the power delivered to the blower (about 70% of the amount of power delivered by the blower when spraying a single liquid through the three nozzles of the conventional single-liquid spray nozzle assembly), the dispersing angles β₁ and β₂ of the spray patterns of the first and second liquid compositions were about 20° to 30°. The concurrent, simultaneous, and divergent spraying of the first and second liquid compositions resulted in side-by-side and divergent dispersed patterns of droplets of the first and second liquid, with detection of no or negligible droplets of mixed composition within the airspace to a distance of at least about 10 feet (3.3 meters) from the spray nozzle apparatus 1. The distribution of the size of the droplets of each of the first and second liquid compositions resulted in the accumulation of coalescing droplets onto the target surfaces along the entire pathway.

### Example 2: Dual Liquid Spraying With Auxiliary Electronic Device

In other non-limiting examples, the blank position 31 in the dual-liquid spray nozzle assembly 4 can be made to secure an auxiliary electronic device, which can be connected to the power and controls of the dual-liquid spray nozzle assembly. In one example, an auxiliary electronic device can be an ultraviolet light lamp, for illuminating the space in front of the dual-liquid spray nozzle assembly with UV light, for providing or assisting sanitizing operations using the into a dual-liquid spray nozzle assembly and apparatus.

In another example, an auxiliary electronic device can be a laser lamp, for emitting a beam of visible or invisible laser light in front of the dual-liquid spray nozzle assembly, to providing a directional signal for the dual-liquid spray nozzle assembly and apparatus.

We claim:

1. A dual-liquid spray apparatus, for dispensing two different liquids, preferably as droplets, concurrently and simultaneously, comprising a spray nozzle assembly comprising at least two nozzles, each nozzle having a nozzle axis for dispersing a spray pattern of a liquid along the nozzle axis, wherein the nozzle axis of a first nozzle for dispersing a first liquid composition diverges at a diverging angle from, and is not parallel to, the nozzle axis of a second nozzle for dispersing a second liquid composition, a first means for supplying the first liquid composition to the first nozzle, and a second means for supplying the second liquid composition to the second nozzle.

2. The dual-liquid spray apparatus according to claim 1, wherein the diverging angle is at least 3 degrees, and preferably at least 10 degrees.

3. A spray nozzle assembly comprising at least two nozzles, each nozzle having a nozzle axis for dispersing a spray pattern of a liquid along the nozzle axis, wherein the nozzle axis of the first nozzle for dispersing a first liquid composition diverges at a diverging angle from, and is not parallel to, the nozzle axis of the second nozzle for dispersing a second liquid composition.

4. The dual-liquid spray apparatus according to claim 3, wherein the diverging angle is at least 3 degrees, and preferably at least 10 degrees.

5. A dual-liquid spray apparatus, for dispensing two different liquids, preferably as droplets, concurrently and alternatingly, comprising a spray nozzle assembly comprising at least one nozzle having a nozzle axis for dispersing a spray pattern of a liquid along the nozzle axis, a first means for supplying a series of pulses of a quantity of a first liquid composition to the at least one nozzle, wherein the series of pulses of the first liquid composition are separated by a first dwell time between successive pulses of the first liquid composition, and a second means for supplying a series of pulses of a quantity of a second liquid composition to the at least one nozzle, wherein the series of pulses of the second liquid composition are supplied during only the first dwell time of the first liquid composition.

6. The dual-liquid spray apparatus according to claim 5, wherein the at least one nozzle comprises a first nozzle and a second nozzle, and the first supply means supplies the first liquid composition to the first nozzle, and the second supply means supplies the second liquid composition to the second nozzle.

7. The dual-liquid spray apparatus according to claim 6, wherein the nozzle axis of the first nozzle diverges at a diverging angle from, and is not parallel to, the nozzle axis of the second nozzle.
